SEQUENCE LISTING (10 sequences)

SEQ ID NO: 1 - DNA, 3708 bp, Mus musculus
gcggcggcggctcctccgggtggtcggctccctcccatctcggccggcccgggcccgtctcgccccccagaaactcactgttcggggctgtggactttctcatcgtgccccacgaaagtaaatcgggggagactgggggagccggaagtaccgcctcgagatccccccaaatacgatcggggaaacggaagtggcccttggtgccaggttgggggagaccggaagtgacgagagctgtcggccatggagccgagtgatagtgccagtaccgctatggaggagcctgacagcctggaggtactggtgaagaccctggactctcagactcggacttttattgtgggggcccagatgaatgtaaaggagtttaaggaacacatagctgcctctgtcagcatcccttccgagaaacagcggctcatctaccagggccgggtcctacaagacgacaagaagctccaggagtacaacgttgggggaaaggttattcacctggtggaacgggctcctcctcagactcagcttccttctggagcatcttctgggacaggctctgcctcagcaactcatggtggggcacccctgcctggcactcggggccctggggcttctgttcatgaccggaatgccaacagctatgtcatggttggaaccttcaatcttcctagtgacggctctgctgtggatgttcacatcaacatggaacaggccccaattcagagtgagccccgggtccggctggtgatggctcagcacatgattagggatatacagaccttactgtcccggatggagtgtcgagggggaacccaagctcaggccagtcagccacccccgcagacaccgcagactgtggcctcagagacggtagccttgaactcacaaacatcagaaccagtcgaaagtgaagctcctcctcgagagcccatggagtcagaagaaatggaggaacgtcccccaacccagactccagagcttgcgccctcaggcccagctcccgcaggcccagctcccgcaggcccagctcccgcgccagagacaaatgcacccaaccacccttcccccgcagagcacgtggaggtgctccaggagcttcagcgcttgcagcgccgtcttcagcctttcctacagcgctactgcgaggtcctgggcgctgctgccaccacagactacaacaacaaccatgagggccgtgaggaggaccagaggttgatcaacttggttggggagagcctgcggctactgggcaacactttcgtggcattgtctgatctgcgctgcaatctagcctgtgcacccccacggcacctgcacgtggttcggcctatgtctcactacacgactcccatggtgctccagcaggcagccattcccattcagatcaatgtggggactactgtgaccatgacaggcaatggggctcggcctccaccagctcctggtgcggaggcagcaaccccaggttctgcccaggccacatccctgcctccctcttccaccactgttgattcatcaactgaaggagctcccccaccagggccagcaccgccaccagcctccagccacccacgggtcatccggatttcccaccagagtgtggagcccgtggtcatgatgcacatgaacattcaagattctggagcacagcctggtggtgtgccaagtgctcccactggtccactgggacctcctggtcatggacagaccctgggacagcaggtgcccggcttcccaacagcaccaactcgggtggtgattgcccggccaactcctccacaggctcggccttcccatcctgggggacctccagtctctggagctctgcagggcgctgggctgggtacaaacacttcattggcccagatggtgagcggccttgtggggcaacttcttatgcagcctgttcttgtggctcaggggactccaggaatggctcaggctcaggcccaggcccaggctcaggcccaggcccaggcccaggctccagctccggctccggctccagctccagctcctgccactgcttcagctagtgctggtactaccaacacagctaccacagctggccctgctcctgggggtcctgcccagcctccacctcctcagccctctgcagctgatcttcagttctctcagctcctgggaaatctgctggggcctgcagggccaggggctggggggccaggcatggcttctcccaccatcactgtggcaatgcctggtgtccctgcttttctccagggcatgactgattttttgcaggcatcacagactgcccctccaccccctccacctcctccacccccaccccctgccccagagcagcagagcacacccccaccagggtctccttctggtggaacagcaagccctggaggcttaggtcctgagagcctgccaccagagtttttcacctcagtggtgcagggcgtgttgagctccctcctgggctccttgggggctcgagctggcagcagtgagagtatcgctgccttcatccaacgcctcagtggatccagcaacatctttgagcctggggctgatggcgcccttggattcttcggagctctgctctctctcctgtgccagaatttctccatggtggatgtggtgatgcttctccatggccatttccagccactgcagcggctccagccacagctgcgatctttcttccaccagcactacctgggtggccaggagcccacgcctagcaacatccggatggcgacccacacactgatcactgggctggaagagtatgtaagggagagtttttctttggtacaggttcagccaggtgtggatatcatccggacaaatttagaatttctccaagagcagtttaacagcattgctgctcatgtgctgcactgtacagacagtggatttggagcccggttgctggagctgtgtaaccagggcctgtttgaatgcttggccctgaacctccactgcttggggggacagcaaatggagcttgctgctgtcatcaatggtcgaattcgccgcatgtctcgcggggtgaatccatccttggtgagctggctgacaaccatgatgggactgaggcttcaggtggtcttggagcacatgcctgtgggtcccgacgccatcctcagatatgttcgtagggttggtgatcctcctcagacacttcctgaagagccgatggaagttcagggagcagaaagaacttcccctgaacctcagagagagaatgcttccccagcccctggaacaacagcagaagaagccatgtcccgaggcccgccccctgctcctgaaggaggttcccgagatgaacaggatggagcttcagctgatgcagaaccctgggcagctgcagtcccccctgaatgggtccctattatccagcaggacattcagagccagcggaaggtgaaacctcagccgcccctgagtgatgcctacctcagtggcatgcctgccaagagacgaaagacaatgcagggtgagggcccccagctgctactctcagaggcagtgagccgggcagctaaggcagccggagctcggcccctgacaagccccgagagcctgagccgggacctggaggcaccagaggttcaggagagctacaggcagcagctccggtctgatatccaaaaacgactgcaggaagatcccaactacagcccccagcgcttccctaatgcccatcgggcatttgctgatgacccctag

SEQ ID NO: 2 - protein, 1154 aa, Mus musculus
MEPSDSASTAMEEPDSLEVLVKTLDSQTRTFIVGAQMNVKEFKEHIAASVSIPSEKQRLIYQGRVLQDDKKLQEYNVGGKVIHLVERAPPQTQLPSGASSGTGSASATHGGAPLPGTRGPGASVHDRNANSYVMVGTFNLPSDGSAVDVHINMEQAPIQSEPRVRLVMAQHMIRDIQTLLSRMECRGGTQAQASQPPPQTPQTVASETVALNSQTSEPVESEAPPREPMESEEMEERPPTQTPELAPSGPAPAGPAPAGPAPAPETNAPNHPSPAEHVEVLQELQRLQRRLQPFLQRYCEVLGAAATTDYNNNHEGREEDQRLINLVGESLRLLGNTFVALSDLRCNLACAPPRHLHVVRPMSHYTTPMVLQQAAIPIQINVGTTVTMTGNGARPPPAPGAEAATPGSAQATSLPPSSTTVDSSTEGAPPPGPAPPPASSHPRVIRISHQSVEPVVMMHMNIQDSGAQPGGVPSAPTGPLGPPGHGQTLGQQVPGFPTAPTRVVIARPTPPQARPSHPGGPPVSGALQGAGLGTNTSLAQMVSGLVGQLLMQPVLVAQGTPGMAQAQAQAQAQAQAQAQAPAPAPAPAPAPATASASAGTTNTATTAGPAPGGPAQPPPPQPSAADLQFSQLLGNLLGPAGPGAGGPGMASPTITVAMPGVPAFLQGMTDFLQASQTAPPPPPPPPPPPPAPEQQSTPPPGSPSGGTASPGGLGPESLPPEFFTSVVQGVLSSLLGSLGARAGSSESIAAFIQRLSGSSNIFEPGADGALGFFGALLSLLCQNFSMVDVVMLLHGHFQPLQRLQPQLRSFFHQHYLGGQEPTPSNIRMATHTLITGLEEYVRESFSLVQVQPGVDIIRTNLEFLQEQFNSIAAHVLHCTDSGFGARLLELCNQGLFECLALNLHCLGGQQMELAAVINGRIRRMSRGVNPSLVSWLTTMMGLRLQVVLEHMPVGPDAILRYVRRVGDPPQTLPEEPMEVQGAERTSPEPQRENASPAPGTTAEEAMSRGPPPAPEGGSRDEQDGASADAEPWAAAVPPEWVPIIQQDIQSQRKVKPQPPLSDAYLSGMPAKRRKTMQGEGPQLLLSEAVSRAAKAAGARPLTSPESLSRDLEAPEVQESYRQQLRSDIQKRLQEDPNYSPQRFPNAHRAFADDP

SEQ ID NO: 3 - DNA, 3702 bp, Homo sapiens
ctcctcggggtgctcggctccctcccacctaggccggccccggcccgactcgccctcagaaactcactgtttggggctgcggactttctcgtcgtgccccacaaaagtaaagcttggggacctggggggagccggaagtatcgcttcgagatccccaaatactatcggggaaacggaagtggccgtcggtggcagagacctgtcggccatggagcctaatgatagtaccagtaccgctgtggaggagcctgacagcttggaggtgttggtgaagaccttggactctcaaactcgtacctttattgtgggggcccagatgaatgtaaaagagtttaaggagcacattgctgcctctgtcagcatcccatctgaaaaacaacggctcatttaccagggacgagttctgcaagatgataagaagcttcaggaatacaatgttgggggaaaggttatccacctggtggaacgggctcctcctcagactcacctcccttctggggcatcttctgggacggggtctgcctcagccactcatggtgggggatccccccctggtactcgggggcctggggcctctgttcatgaccggaatgccaacagctatgtcatggttggaaccttcaatcttcctagtgacggctctgctgtggatgttcacatcaacatggaacaggccccgattcagagtgagccccgggtacggctggtgatggctcagcacatgatcagggatatacagaccttactatcccggatggagtgtcgaggagggccccaaccgcagcacagtcagccgcccccgcagccaccggctgtgaccccggagccagtagccttgagctctcaaacatcagaaccagttgaaagtgaagcacctccccgggagcccatggaggcagaagaagtggaggagcgtgccccagcccagaacccggagctcactcctggcccagccccagcgggcccaacacctgccccggaaacaaatgcacccaaccatccttcccctgcggagtatgtcgaggtgctccaggagctacagcggctggagagtcgcctccagcccttcttgcagcgctactacgaggttctgggtgctgctgccaccacggactacaataacaatcacgagggccgggaggaggatcagcggttgatcaacttggtaggggagagcctgcgactgctgggcaacacctttgttgcactgtctgacctgcgctgcaatctggcctgcacgcccccacgacacctgcatgtggtccggcctatgtctcactacaccacccccatggtgctccagcaggcagccattcccatacagatcaatgtgggaaccactgtgaccatgacaggaaatgggactcggccccccccaactcccaatgcagaggcacctccccctggtcctgggcaggcctcatccgtggctccgtcttctaccaatgtcgagtcctcagctgagggggctcccccgccaggtccagctcccccgccagccaccagccacccgagggtcatccggatttcccaccagagtgtggaacccgtggtcatgatgcacatgaacattcaagattctggcacacagcctggtggtgttccgagtgctcccactggccccctgggaccccctggtcatggccaaaccctgggacagcaggtgccaggcttcccaacagctccaacccgggtggtgattgcccggcccactcctccacaggctcggccttcccatcctggagggcccccagtctctgggacactgcagggcgccggtctgggtaccaatgcctcgttggcccagatggtgagcggccttgtggggcagcttcttatgcagccagtccttgtggctcaggggaccccaggtatggctccaccgccagcccctgccactgcttctgccagtgctggcaccaccaacacagctaccacagctggccccgctcctggggggcctgcccagcctccacccacccctcaaccctccatggctgatcttcagttctctcagcttctggggaacctgctagggcctgcagggccaggggctggagggtctggtgtggcttctcccaccatcactgtggcgatgcctggtgtccctgcctttctccaaggcatgactgacttcttgcaggcaacacagacagcccctccaccacccccacctcctccacccccaccacctgccccagagcagcagaccatgcccccaccaggctccccttctggtggcgcagggagtcctggaggcctgggtcttgagagcctgtcaccggagttttttacctcagtggtgcagggtgtgctcagctccctgctgggctccctgggggctcgggctggcagcagtgaaagtattgctgccttcatacaacgcctcagtggatccagcaacatctttgagcctggagctgatggggcccttggattctttggggccttgctttctcttctgtgccagaacttctctatggtggacgtagtgatgcttctccatgggcatttccagccactacaacggctccagccccagctgcgatccttcttccaccagcactacctgggtggtcaggagcccacacccagtaacatccggatggcaacccacacattgatcacggggctagaagagtatgtgcgggagagtttttccttggtgcaggttcagccaggtgtggacatcatccggacaaacctggaatttctccaagagcagtttaatagcattgctgcgcatgtgctgcattgcacagatagtggatttggggcccggttgctggagttgtgtaaccaaggcctgtttgaatgcctggccctaaacctgcactgcttggggggacagcagatggagcttgctgctgttatcaatggccgaattcgtcgtatgtctcgtggggtgaatccctccttggtgagctggctgaccactatgatgggactgaggcttcaggtggtactggagcacatgcctgtaggccctgatgccattctcagatacgttcgcagggttggtgatcccccccagccacttcctgaggagccaatggaagttcagggagcagaaagagcttcccctgagcctcagcgggagaatgcttccccagcccctggaacaacagcagaagaggccatgtcccgaggtccacctcctgctcctgaggggggctcccgggatgaacaggatggagcttcagctgagacagaaccttgggcagctgcagtccccccagaatgggtccctattatccagcaggacattcagagccagcggaaggtgaaaccgcagccccctctgagtgatgcctacctcagtggtatgcctgccaagagacgcaagacgatgcagggtgagggcccccagctgcttctctcagaggctgtgagccgggcagctaaggcagccggagctcggcccctgacgagccccgagagcctgagccgggacctggaggcaccagaggttcaggagagctacaggcagcagctccggtctgatatacaaaaacgactgcaggaagaccccaactacagtccccagcgcttccccaatgcccagcgggcctttgctgatgatccttagctctttgctctatggcccttcctcatcaggggaccgtttcccccctcttccttcacagtatttaagaaataaaagtcggatttttctggcaaaaaaaaaaaaaaaaaaaaaaa

SEQ ID NO: 4 - protein, 1126 aa, Homo sapiens
MEPNDSTSTAVEEPDSLEVLVKTLDSQTRTFIVGAQMNVKEFKEHIAASVSIPSEKQRLIYQGRVLQDDKKLQEYNVGGKVIHLVERAPPQTHLPSGASSGTGSASATHGGGSPPGTRGPGASVHDRNANSYVMVGTFNLPSDGSAVDVHINMEQAPIQSEPRVRLVMAQHMIRDIQTLLSRMECRGGPQPQHSQPPPQPPAVTPEPVALSSQTSEPVESEAPPREPMEAEEVEERAPAQNPELTPGPAPAGPTPAPETNAPNHPSPAEYVEVLQELQRLESRLQPFLQRYYEVLGAAATTDYNNNHEGREEDQRLINLVGESLRLLGNTFVALSDLRCNLACTPPRHLHVVRPMSHYTTPMVLQQAAIPIQINVGTTVTMTGNGTRPPPTPNAEAPPPGPGQASSVAPSSTNVESSAEGAPPPGPAPPPATSHPRVIRISHQSVEPVVMMHMNIQDSGTQPGGVPSAPTGPLGPPGHGQTLGQQVPGFPTAPTRVVIARPTPPQARPSHPGGPPVSGTLQGAGLGTNASLAQMVSGLVGQLLMQPVLVAQGTPGMAPPPAPATASASAGTTNTATTAGPAPGGPAQPPPTPQPSMADLQFSQLLGNLLGPAGPGAGGSGVASPTITVAMPGVPAFLQGMTDFLQATQTAPPPPPPPPPPPPAPEQQTMPPPGSPSGGAGSPGGLGLESLSPEFFTSVVQGVLSSLLGSLGARAGSSESIAAFIQRLSGSSNIFEPGADGALGFFGALLSLLCQNFSMVDVVMLLHGHFQPLQRLQPQLRSFFHQHYLGGQEPTPSNIRMATHTLITGLEEYVRESFSLVQVQPGVDIIRTNLEFLQEQFNSIAAHVLHCTDSGFGARLLELCNQGLFECLALNLHCLGGQQMELAAVINGRIRRMSRGVNPSLVSWLTTMMGLRLQVVLEHMPVGPDAILRYVRRVGDPPQPLPEEPMEVQGAERASPEPQRENASPAPGTTAEEAMSRGPPPAPEGGSRDEQDGASAETEPWAAAVPPEWVPIIQQDIQSQRKVKPQPPLSDAYLSGMPAKRRKTMQGEGPQLLLSEAVSRAAKAAGARPLTSPESLSRDLEAPEVQESYRQQLRSDIQKRLQEDPNYSPQRFPNAQRAFADDP

SEQ ID NO: 5 - DNA, 21 bp, Artificial Sequence
  Synthetic oligonucleotide Bat 3
cagctccggtctgatatacaa

SEQ ID NO: 6 - DNA, 21 bp, Artificial Sequence
  Synthetic oligonucleotide Bat 3*
atgatgcacatgaacattcaa

SEQ ID NO: 7 - DNA, 30 bp, Artificial Sequence
  Synthetic oligonucleotide  5′ primer NTPR
cggatccccatggagacatacaagtccaac

SEQ ID NO: 8 - DNA, 27 bp, Artificial Sequence
  Synthetic oligonucleotide  3′ primer NTPR
gctcgagtcagttgtcggggtccagct

SEQ ID NO: 9 - DNA, 24 bp, Artificial Sequence
  Synthetic oligonucleotide  5′ primer TPRC
atggatccatgcccgcgcgaaccg

SEQ ID NO: 10 - DNA, 30 bp, Artificial Sequence
  Synthetic oligonucleotide  3′ primer TPRC
gctcgagtcactcctgctggtcgtcgttgc

(19) United States
(12) Patent Application Publication
(10) Pub. No.: US 2009/0226446 A1
(43) Pub. Date: Sep. 10, 2009

(54) Method to Inhibit the Propagation of an Undesired Cell Population

(75) Inventors: Celina Cziepluch, Heidelberg (DE); Jean Rommelaere, Heidelberg (DE); Marc Winnefeld, Heidelberg (DE)

Correspondence Address: BIRCH STEWART KOLASCH & BIRCH, PO BOX 747, FALLS CHURCH, VA, 22040-0747, US

(73) Assignee: Deutsches Krebsforschungszentrum Stiftung des offentilchen Rechts, Heidelberg (DE)

(21) Appl. No.: 12/226,013

(22) PCT Filed: Apr. 5, 2007
(86) PCT No.: PCT/EP2007/053403

§ 371 Date: Oct. 3, 2008

Related U.S. Application Data
(60) Provisional application No. 60/789,600, filed on Apr. 6, 2006.

(51) Int. Cl. A61K 39/395; C12N 5/08; C12N 5/06; A61K 31/7105; A61K 38/02; C12Q 1/02; G01N 33/53

(52) U.S. Cl. 424/139.1; 435/366; 435/375; 514/44 A; 514/2; 435/29; 435/7.2

(57) ABSTRACT

Disclosed is a method for inhibiting the propagation of an undesired cell population, the method comprising introducing into a cell an antagonist of Bat 3, or of a functional variant thereof, said antagonist depleting Bat 3, a functional variant thereof, in a cell of said cell population, and verifying the reduced proliferation and/or cell death in Bat 3 depleted cell populations; disclosed is further the use of a Bat 3 antagonist for the manufacture of a medicament for the treatment of diseases which are caused by the propagation of an undesired cell population.

### FIELD

The present invention refers to a method to eliminate an undesired cell population, particularly aberrantly growing tumor cells, by specifically inactivating or depleting the HLA-B-associated transcript 3 (Bat 3).

### BACKGROUND OF THE INVENTION

Cell division and proliferation is a normal ongoing process in all living organisms and involves numerous factors and signals that are delicately balanced to maintain regular cellular cycles. The general process of cell division consists of two sequential processes: nuclear division (mitosis), and cytoplasmic division (cytokinesis). Because organisms are continually growing and replacing cells, cellular proliferation is vital to the normal functioning of almost all biological processes. Whether or not mammalian cells will grow and divide is determined by a variety of feedback control mechanisms, which include the availability of space in which a cell can grow, and the secretion of specific stimulatory and inhibitory factors in the immediate environment. When normal cellular proliferation is disturbed or somehow disrupted, the results can affect an array of biological functions. Disruption of proliferation could be due to a myriad of factors such as the absence or overabundance of various signaling chemicals or presence of altered environments. Disorders characterized by abnormal cellular proliferation include cancer, abnormal development of embryos, improper formation of the corpus luteum, difficulty in wound healing as well as malfunctioning of inflammatory and immune responses.

Abnormally proliferating cancer cells exhibit a number of properties that make them dangerous to the host, often including an ability to invade other tissues. One of the defining features of cancer cells is that they respond abnormally to control mechanisms that regulate the division of normal cells and continue to divide in a relatively uncontrolled fashion until they kill the host. The effective cure of patients, though, is often difficult since many tumor cells develop a resistance to radiation therapy or cytostatic drugs used for chemotherapy.

The described phenotype, also termed drug resistance, involves a variety of strategies that tumor cells use to evade the cytostatic or cytolytic effects of anticancer drugs. Mechanisms for drug resistance include modifications in detoxification and DNA repair pathways, changes in cellular sites of drug sequestration, decreases in drug-target affinity, synthesis of specific drug inhibitors within cells, and accelerated removal or secretion of drugs. More recently, cellular drug resistance has been associated with alterations in the so-called programmed cell death, a process known as apoptosis. Thus, many methods of the prior art which are employed to solve the problem of successfully circumventing drug-resistance rely on compounds or methods which directly affect, i.e. enhance, various steps in apoptosis. Other approaches include, for example, the up- or down-regulation of distinct genes which have demonstrated to be deregulated in drug-resistant tumor cells.

In view of the increasing number in cancer cases, any approach to improve current treatment methods is of important ethic, social and economic value. Furthermore, a variety of other circumstances like e.g. inflammatory diseases or viral infections can make it necessary to eliminate cells which are infected by various microorganisms.

### SUMMARY OF THE INVENTION

In a first embodiment, the present invention pertains to a method for inhibiting the propagation of an undesired cell population, the method comprising
(i) introducing an antagonist of the HLA-B-associated transcript 3 (Bat 3) into at least one cell of said cell population, and
(ii) cultivating said cell population for a time period sufficient to allow said Bat 3 antagonist to be effective, thereby inactivating and/or depleting Bat 3 in said cell population.

In a preferred embodiment of the method of the present invention, the cell population is in the mitotic stage.

In another preferred embodiment of the method of the present invention, the cell population is in a resting stage.

In a furthermore preferred embodiment of the method of the present invention, the cell population is a population of human cells.

In another preferred embodiment of the method of the present invention, the antagonist is selected from the group consisting of a Bat 3-specific siRNA, a transcriptional regulator of the Bat 3 gene, a Bat 3 gene antisense molecule, a Bat 3 mRNA specific ribozyme, an antibody against a Bat 3 polypeptide, a Bat 3-specific aptamer and a Bat 3-specific mutein.

In a furthermore preferred embodiment of the method of the present invention, the antagonist is a Bat 3-specific siRNA. Most preferably, the siRNA comprises a sequence as defined by SEQ ID NO: 5 or 6.

The present invention also pertains to a method of treating a subject suffering from a disease caused by the propagation of an undesired cell population comprising administering to said subject a therapeutically effective amount of a Bat 3-antagonist, optionally in combination with a pharmaceutically acceptable carrier.

In a preferred embodiment of the method of the present invention, the disease is cancer.

In a preferred embodiment of the method of the present invention, the cancer disease is selected from the group consisting of neuroblastoma, intestine carcinoma, rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroid carcinoma, papillary thyroid carcinoma, follicular thyroid carcinoma, anaplastic thyroid carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors, glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeolid leukemia (AML), chronic myeloid leukemia (CML), adult T-cell leukemia lymphoma, hepatocellular carcinoma, gall bladder carcinoma, bronchial carcinoma, multiple myeloma, basalioma, teratoma, retinoblastoma, neuroblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma and plasmocytoma.

In another preferred embodiment of the method of the present invention, the disease is an autoimmune disease.

In another preferred embodiment of the method of the present invention, the Bat 3-antagonist is selected from the group consisting of a Bat 3-specific siRNA, a transcriptional regulator of the Bat 3 gene, a Bat 3 gene antisense molecule, a Bat 3 mRNA specific ribozyme, an antibody against a Bat 3 polypeptide, a Bat 3-specific aptamer and a Bat 3-specific mutein.

In a further preferred embodiment of the method of the present invention, the Bat 3-antagonist is a Bat 3-specific siRNA. Preferably, the siRNA comprises a sequence as defined in SEQ ID NO 5 or 6.

The present invention also pertains to a method for screening candidate compounds for at least one Bat 3 antagonist with the ability to inhibit the propagation of a cell population, the method comprising the following steps:
(i) contacting a cell population with a candidate compound, thereby enabling the introduction of said candidate compound into the cells of said cell population,
(ii) cultivating said cell population for a time period sufficient to allow the candidate compound to be effective, and parallel cultivating a control cell population which has not been contacted with the candidate compound, and
(iii) monitoring cell growth and/or cell properties in said cell population and in the control cell population,
   wherein a reduced growth and/or altered cell properties as compared to the control cell population is indicative that the candidate compound is an Bat 3 antagonist which inhibits the propagation of a cell population.

In a preferred embodiment of the method of the present invention, the method comprises the additional steps:
(iv) qualitatively and/or quantitatively detecting Bat 3 expression levels in said cell population and in the control cell population, wherein a lower level of Bat 3 expression is indicative of a compound that is a Bat 3 antagonist, and
(v) determining whether a lower level of Bat 3 expression correlates with a reduced growth and/or altered cell properties of the cell population being contacted with the candidate compound.

In another preferred embodiment of the method of the present invention, the cell population is in a mitotic stage.

In a further preferred embodiment of the method of the present invention, the cell population is a population of human cells.

The present invention also relates to a method for screening candidate compounds for at least one Bat 3-antagonist with the ability to inhibit the propagation of a cell population comprising:
(i) contacting a Bat 3-polypeptide and a h-SGT polypeptide with a candidate compound for a Bat 3-antagonist; and
(ii) determining whether the candidate compound is capable of blocking the interaction of Bat 3 and h-SGT, whereby a Bat 3-antagonist is identified.

In a preferred embodiment of the method of the present invention, the Bat 3-polypeptide and the h-SGT polypeptide are comprised by a cell.

In a more preferred embodiment of the method of the present invention, the cell is in a mitotic stage and, most preferably, the Bat 3-antagonist blocks progression of mitosis.

In a further preferred embodiment of the method of the present invention, the Bat 3-antagonist is selected from the group consisting of: a small molecule, an antibody against a Bat 3-polypeptide, a Bat 3-specific aptamer and a Bat 3-specific mutein.

The present invention also relates to a method for the preparation of a pharmaceutical composition, wherein a Bat 3-antagonist inhibiting the propagation of an undesired cell population is identified according to any one of the previous methods and synthesized in adequate amounts and formulated into a pharmaceutical composition.

Finally, the present invention encompasses a medicament comprising a Bat 3-antagonist in a therapeutically effective amount.

### DESCRIPTION OF THE DRAWINGS

FIG. 1: (A) hSGT-binding partners identified by MALDI-TOF mass-spectrometry analysis were verified by co-immunoprecipitation and subsequent immunoblotting. Co-immunoprecipitation were performed with extracts from interphase cells expressing Flag-hSGT protein using either a Flag-tag-specific monoclonal antibody (lane 2) or a control antibody IVA7 (lane 1). Flag-hSGT indicates the position of hyper- and hypo-phosphorylated forms of Flag-hSGT. Lane 3 and 4 represent a fraction ( 1/100) of the unbound proteins present in the supernatant of the respective co-immunoprecipitation reactions with IVA7 or Flag-tag-specific antibodies. (B) Schematic depiction of the Flag-tagged hSGT proteins expressed in vivo for interaction mapping. (C) Mapping of the interaction regions within hSGT was performed using extracts from cells expressing Flag-tagged versions of either full-length (lane 1 and 2), N-terminally truncated (TPRC) (lane 3 and 4) or C-terminally truncated (NTPR) (lane 5 and 6) hSGT polypeptides. Co-immunoprecipitation experiments were performed using either control IVA7 (lane 2, 4 and 6) or Flag-tag-specific antibodies (lane 1, 3 and 5). Precipitated polypeptides were fractionated by SDS-PAGE and analysed through immunoblotting using antibodies recognizing either Hsc70, Hsp70, Bat 3 or the Flag-tag.

FIG. 2: (A) Bat 3 protein levels in HeLa H2A-YFP cells, 24 h and 42 h after treatment with either of two independent Bat 3-specific siRNAs, Bat 3 (SEQ ID NO: 5) or Bat 3* (SEQ ID NO: 6). Extracts from equal amounts of cells were loaded per lane and Bat 3 or Hsc70 and Hsp70 (loading control) were detected by immunoblotting. (B a) Time-lapse video microscopic images of HeLa H2A-YFP cell populations with reduced levels of Bat 3 revealed many cells (arrows) in prometaphase with few misaligned chromosomes while most chromosomes were aligned in the metaphase plate. Cells were analysed by phase contrast and fluorescence (YFP) microscopy between 24 and 54 h post-transfection. (B b) Bat 3-depleted cells showed a prolonged prometaphase with few continuously misaligned chromosomes visualized through the YFP fluorescence of histon 2A (e.g. cell marked with arrowhead in overview. B a is depicted in B b). This cell (B b) died in prometaphase without rescuing the misaligned chromosomes. Time is given in hours and minutes (h:min).

### DETAILED DESCRIPTION OF THE INVENTION

The object of the present invention is to provide means and methods which, in principle, allow eliminating cells which are abnormally growing, inflamed, infected or otherwise altered, making it desirable to eliminate the affected cell population.

Therefore, the present invention relates to a method for inhibiting the propagation of an undesired cell population, the method comprising
(i) introducing an antagonist of Bat 3 into at least one cell of said cell population, and
(ii) cultivating said cell population for a time period sufficient to allow said antagonist to be effective, thereby inactivating and/or depleting Bat 3 in said cell population
   Preferably, the method of the present invention comprises an additional step, which can be:
(iii) monitoring the growth and/or cell properties of said cell population,
   wherein a reduced growth and/or altered cell properties of said cell population as compared to the control cell population is indicative that the propagation of the undesired cell population has been successfully inhibited.

Preferably, the method of the present invention, in particular step (ii), can be performed such that a control cell population which has not being contacted with the Bat 3 antagonist is cultivated in parallel, allowing for a comparison of growth and/or cell properties between the cell population which has been contacted and the cell population which has not been contacted with the Bat 3 antagonist.

The method of the present invention may be carried out in vitro, i.e. by using isolated cells, cell lines, tissues or organs, or in vivo, i.e. as a method of treating an organism by inhibiting the propagation of an undesired cell population.

Preferably, the method of the present invention is performed in vitro.

Monitoring growth and/or cell properties can occur by several methods known to the person skilled in the art. It includes determining the number of living and/or dead cells within the population, determining the adherence of the cells to the culture dish, determining the shape of the cell, with a detachment and round-up indicating cell death and determining the cell cycle stage of the cell population.

In the context of the present invention, the term “undesired cell population” includes, among others, cell populations which are abnormally dividing, e.g. tumor/cancer cells, cells infected with microorganisms such as bacteria, viruses or yeasts, cells which are affected by toxic substances and autoreactive cells of the immune system. Preferably, the term “an undesired cell population” refers to tumor/cancer cells.

In a preferred embodiment, the cell population which is subjected to the method of the present invention is in the mitotic stage. It has been found that Bat 3 particularly acts during mitosis and a depletion of Bat 3 predominantly prevents cells from properly undergoing mitosis.

In a preferred embodiment of the present invention the cell population is a cell population of mammalians, such as humans, non-human primates, dogs, cats, cattle, horses, sheep, and the like. Further encompassed are cell populations from insects, nematodes, fish or yeast cell populations.

More preferably, the method of the present invention is performed by the depletion of human Bat 3. Consequently, the undesired mammalian cell population is preferably a population of human cells.

The term “HLA-B-associated transcript 3” or “Bat 3” relates, preferably, to a polynucleotide comprising a nucleic acid sequence as disclosed in Wang 1994, Mol. Cell. Biochem. 136(1): 49-57 or as deposited under GeneBank Accession no. NM_{—}057171.1, GI:33147081 (SEQ ID NO:1) or to the polypeptide encoded by said sequence (SEQ ID NO: 2). The term also encompasses the sequence disclosed in GeneBank Accession no. BC003133.1, GI: 13111924, see also Strausberg, 2002, Proc. Natl. Acad. Sci. USA 99 (26): 16899-16903 (SEQ ID NO: 3) or the polypeptide encoded thereby (SEQ ID NO: 4). The term as used herein, thus refers dependent on the context to both, either the amino acid sequence of the polypeptide or the nucleic acid sequence of the polynucleotide. The designations “Bag-6” and “Scythe” are synonymous designations for Bat 3 which may be used in the prior art and herein below. It is to be understood that the term further encompasses functional variants of the aforementioned specific polynucleotides and polypeptides. Said functional variants may be allelic variants, homologs, orthologs or paralogs, the depletion of which results in an inhibition of the proliferation of an undesired cell population as specified herein. Moreover, depending on the context, i.e. whether Bat 3 refers to a polypeptide or a polynucleotide, the mode of action for antagonists depleting or inactivating Bat 3 can greatly differ. Details are given below in this specification.

The term “functional variant” of the Bat 3 polypeptide includes peptides in which one or more amino acids of the original Bat 3 sequence can be substituted by one or more amino acids different from the original one(s), or peptides the amino acid sequence of which is extended or shorterned on the aminoterminal and/or the carboxyterminal end or internally, and which still show the proposed effect of inhibiting the propagation of an undesired cell population. Preferably, a functional variant has an amino acid sequence being at least 50%, 60%, 70%, 80%, 90%, 95%, 97%, 98%, or 99% identical with the aforementioned specific amino acid sequences.

The term “functional variant” of the Bat 3 nucleic acid includes nucleic acids in which one or more nucleotides of the original Bat 3 DNA sequence can be substituted by one or more nucleotides different from the original one(s), or nucleic acids the nucleotide sequence of which is extended or shortened on the 5′-terminal and/or the 3′-terminal end or internally, and which still show the proposed effect of inhibiting the propagation of an undesired cell population. Preferably, a functional variant has an nucleic acid sequence being at least 50%, 60%, 70%, 80%, 90%, 95%, 97%, 98%, or 99% identical with the aforementioned specific nucleic acid sequences.

Percentage of sequence identity is, preferably, determined by comparing two optimally aligned sequences over a comparison window (preferably windows of at least 100 nucleotides or at least 10 amino acids), where the fragment of the polynucleotide or amino acid sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by inspection. Given that two sequences have been identified for comparison, GAP and BESTFIT are preferably employed to determine their optimal alignment. Typically, the default values of 5.00 for gap weight and 0.30 for gap weight length are used.

It is to be understood that if it is referred to a polypeptide or polynucleotide by using the singular terms, such wording is meant to also include plural terms, i.e. it is also referred to a plurality of molecules the said polypeptide or polynucleotide.

The term “antagonist” refers to any compound that is capable of directly depleting the expression and/or the function of Bat 3, either on the nucleic acid level or on the polypeptide level. It is further contemplated within the scope of the present invention that the antagonist refers to any compound that depletes the expression and/or the function of Bat 3 indirectly, e.g. by depleting compounds which are required for proper Bat 3 expression and function.

If the depletion occurs on the nucleic acid level, the antagonist according to the present invention can be a peptide or a nucleic acid that regulates the transcription of the Bat 3 gene by binding to up-stream and/or down-stream regulatory sequences of the coding region of Bat 3. Such regulatory sequences are known to the person skilled in the art and include so-called promoter, operator, enhancer or silencer regions. For example, the Bat 3 antagonist may interfere with the binding of the RNA polymerase to the promoter region of the Bat 3 gene, either by binding directly to the RNA polymerase binding region, by binding to the polymerase itself or by binding to other factors, e.g. transcription factors, which are required for efficient RNA polymerase binding and function. Furthermore, the Bat 3 antagonist may bind to the operator region and act as a so-called repressor of Bat 3 gene expression.

In a further embodiment of the present invention, the depletion on the nucleic acid level can occur by the use of nucleic acid molecules that hybridize to, and are therefore complementary to the coding sequence of Bat 3. These nucleic acid molecules may encode or act as Bat 3 gene antisense molecules useful, for example, in Bat 3 gene regulation. With respect to Bat 3 gene regulation, such techniques can be used to modulate, for example, the phenotype and metastatic potential of cancer cells. The use of antisense molecules as inhibitors is a specific, genetically based therapeutic approach. The present invention provides the therapeutic and prophylactic use of nucleic acids of at least six nucleotides that are antisense to a gene or cDNA encoding Bat 3.

A promising tool to deplete or down-regulate and interfere with the expression of proteins has been developed according to the observation that double-stranded RNA molecules (dsRNA) with homology to a defined sequence within a gene acts as a “trigger” for post-transcriptional gene silencing (PTGS). The unique aspect of this process named “RNA interference” (RNAI) is its exquisite sequence-specificity, leading to the degradation of the targeted mRNA. RNAi has been originally discovered as a mechanism of PTGS in plants and animals. RNAi is a process of sequence-specific, post-transcriptional gene silencing in organisms initiated by double-stranded RNA (dsRNA) that is homologous in sequence to the gene to be silenced. The RNAi technique involves small interfering RNAs (siRNAs) that are complementary to target RNAs (encoding a gene of interest) and specifically destroy the known mRNA, thereby diminishing or abolishing gene expression. RNAi is generally used to silence expression of a gene of interest by targeting mRNA, however, any type of RNA is encompassed by the RNAi methods of the invention. Briefly, the process of RNAi in the cell is initiated by long double stranded RNAs (dsRNAs) being cleaved by a ribonuclease, thus producing siRNA duplexes. The siRNA binds to another intracellular enzyme complex which is thereby activated to target whatever mRNA molecules are homologous (or complementary) to the siRNA sequence. The function of the complex is to target the homologous mRNA molecule through base pairing interactions between one of the siRNA strands and the target mRNA. The mRNA is then cleaved approximately 12 nucleotides from the 3′ terminus of the siRNA and degraded. In this manner, specific mRNAs can be targeted and degraded, thereby resulting in a loss of protein expression from the targeted mRNA. A complementary nucleotide sequence as used herein refers to the region on the RNA strand that is complementary to an RNA transcript of a portion of the target gene. The term “dsRNA” refers to RNA having a duplex structure comprising two complementary and anti-parallel nucleic acid strands. Not all nucleotides of a dsRNA necessarily exhibit complete Watson-Crick base pairs; the two RNA strands may be substantially complementary. The RNA strands forming the dsRNA may have the same or a different number of nucleotides, with the maximum number of base pairs being the number of nucleotides in the shortest strand of the dsRNA. Preferably, the dsRNA is no more than 49, more preferably less than 25, and most preferably between 19 and 23, nucleotides in length. dsRNAs of this length are particularly efficient in inhibiting the expression of the target gene using RNAi techniques. dsRNAs are subsequently degraded by a ribonuclease enzyme into short interfering RNAs (siRNAs). RNAi is mediated by small interfering RNAs (siRNAs). The term “small interfering RNA” or “siRNA” refers to a nucleic acid molecule which is a double stranded RNA agent that is complementary to i.e., able to base-pair with, a portion of a target RNA (generally mRNA), i.e. the polynucleotide of the present invention being RNA. siRNA acts to specifically guide enzymes in the host cell to cleave the target RNA. By virtue of the specificity of the siRNA sequence and its homology to the RNA target, siRNA is able to cause cleavage of the target RNA strand, thereby inactivating the target RNA molecule. Preferably, the siRNA which is sufficient to mediate RNAi comprises a nucleic acid sequence comprising an inverted repeat fragment of the target gene and the coding region of the gene of interest (or portion thereof).

Also preferably, a nucleic acid sequence encoding a siRNA comprising a sequence sufficiently complementary to a target gene is operatively linked to a expression control sequence. Thus, the mediation of RNAi to inhibit expression of the target gene can be modulated by said expression control sequence. Preferred expression control sequences are those which can be regulated by a exogenous stimulus, such as the tet operator whose activity can be regulated by tetracycline or heat inducible promoters. Alternatively, an expression control sequence may be used which allows tissue-specific expression of the siRNA.

The complementary regions of the siRNA allow sufficient hybridization of the siRNA to the target RNA and thus mediate RNAi. In mammalian cells, siRNAs are approximately 21-25 nucleotides in length. The siRNA sequence needs to be of sufficient length to bring the siRNA and target RNA together through complementary base-pairing interactions. The siRNA used with the Tet expression system of the invention may be of varying lengths. The length of the siRNA is preferably greater than or equal to ten nucleotides and of sufficient length to stably interact with the target RNA; specifically 15-30 nucleotides; more specifically any integer between 15 and 30 nucleotides, most preferably 15, 16, 17, 18, 19, 20, 21, 22, 23, 24, 25, 26, 27, 28, 29, and 30. By sufficient length is meant an oligonucleotide of greater than or equal to 15 nucleotides that is of a length great enough to provide the intended function under the expected condition. Stable interaction means interaction of the small interfering RNA with target nucleic acid (e.g., by forming hydrogen bonds with complementary nucleotides in the target under physiological conditions).

In principle, such complementarity is 100% between the siRNA and the RNA target, but can be less if desired, preferably 91%, 92%, 93%, 94%, 95%, 96%, 97%, 98%, or 99%. For example, 19 bases out of 21 bases may be base-paired. In some instances, where selection between various allelic variants is desired, 100% complementary to the target gene is required in order to effectively discern the target sequence from the other allelic sequence. When selecting between allelic targets, choice of length is also an important factor because it is the other factor involved in the percent complementary and the ability to differentiate between allelic differences.

Methods relating to the use of RNAi to silence genes in organisms, including C. elegans, Drosophila, plants, and mammals, are known in the art (see, for example, Fire et al., Nature (1998) 391:806-811; Fire, Trends Genet. 15, 358-363 (1999); Sharp, RNA interference 2001. Genes Dev. 15, 485-490 (2001); Hammond et al. Nature Rev. Genet. 2, 1110-1119 (2001); Tuschl, Chem. Biochem. 2, 239-245 (2001); Hamilton et al., Science 286, 950-952 (1999); Hammond et al., Nature 404, 293-296 (2000); Zamore et al., Cell 101, 25-33 (2000); Bernstein et al., Nature 409, 363-366 (2001); Elbashir et al., Genes Dev. 15, 188-200 (2001); WO 0129058; WO 09932619; and Elbashir et al., 2001 Nature 411: 494-498). RNAi may be used to specifically inhibit expression of the polynucleotides of the present invention in vivo. Accordingly, it may be used for therapeutic approaches for diseases or disorders which are accompanied with an altered expression of the polynucleotides of the present invention, e.g., an enhanced expression or a expression of the polynucleotides at wrong locations. For such therapeutic approaches, expression constructs for siRNA may be introduced into target cells of the host which suffer from altered polynucleotide expression. Accordingly, siRNA may be combined efficiently with the available gene therapy approaches.

Thus, in a preferred embodiment of the present invention, the Bat 3 antagonist of the present invention that depletes the expression and/or the function of a Bat 3 on the nucleic acid level is a dsRNA molecule which is complementary to the Bat 3 mRNA. Preferably, the dsRNA molecules which are complementary to the mRNA of Bat 3 of the present invention have a length between 10 and 30 base pairs, more preferably, they have a length between 19 and 25 base pairs. Most preferred, the siRNA is 21 base pairs in length and corresponds to the sequence as set out in SEQ ID NO: 5 or 6. The Bat 3 antagonist being siRNA may be delivered to the target cell by any method known the one of skilled art. Applicable is, for instance, the delivery by using cationic liposome reagents.

As the effect of siRNAs, i.e. the reduction of the expression of a certain gene, is considered to be only transient when they are directly applied to cells as described supra it can be advantageous if the nucleic acid, preferably a DNA, encoding the respective Bat 3 siRNA is integrated in an expression vector. Providing suitable elements, as described hereinafter, the DNA is transcribed into the corresponding RNA which is capable of forming the desired siRNA.

The expression vector is preferably a eukaryotic expression vector, or a retroviral vector, a plasmid, bacteriophage, or any other vector typically used in the biotechnology field. If necessary or desired, the nucleic acid can be operatively linked to regulatory elements which direct the synthesis of a mRNA in pro- or eukaryotic cells. Such regulatory elements are promoters, enhancers or transcription termination signals, but can also comprise introns or similar elements, for example those, which promote or contribute to the stability and the amplification of the vector, the selection for successful delivery and/or the integration into the host's genome, like regions that promote homologous recombination at a desired site in the genome. For therapeutic purposes, the use of retroviral vectors has been proven to be most appropriate to deliver a desired nucleic acid into a target cell. Preferably, the vector to be used in accordance with the method of the present invention is a parvovirus vector.

The nucleic acid, preferably a DNA, which is suitable for the preparation of a Bat 3 siRNA can also be introduced into a vector which allows for the production of a double-stranded (ds) RNA molecule. Such vectors are known to the person skilled in the art. To drive the expression of siRNAs these vectors usually contain RNA polymerase III promoters, such as the H1 or U6 promoter, since RNA polymerase III expresses relatively large amounts of small RNAs in mammalian cells and terminates transcription upon incorporating a string of 3-6 uridines. Type III promoters lie completely upstream of the sequence being transcribed which eliminates any need to include promoter sequence in the siRNA. If the DNA encoding the desired siRNA should be transcribed from one promoter, the preferred DNA should thus contain the desired coding region of the Bat 3 gene to be targeted as well as its complementary strand, wherein the coding and its complementary strand are separated by a nucleotide linker, allowing for the resulting transcript to fold back on itself to form a so-called stem-loop structure.

An example for such an expression vector which allows for the production of dsRNA directly in the target cell is the so-called pSUPER (Supression of Endogenous RNA). The vector itself and the mechanism how the dsRNA is produced by using said vector is described in Brummelkamp et al., 2002, Science, Vol. 296, pages 550-553. Another example of such a vector named pSilencer (Ambion) was developed by Sui et al., 2002, Proc. Natl. Acad. Sci. Vol. 99, pages 5515-5520.

Furthermore, the present invention encompasses so-called ribozymes as Bat 3 antagonists. Ribozymes are naturally occurring RNA fragments that can be designed as human therapeutics to recognize, bind and digest any disease-causing mRNA sequence, in this case the Bat 3 mRNA. Ribozymes are designed to target the Bat 3 mRNA through complementary base pair hybridization. After binding to the target, the enzymatic activity of the ribozyme cleaves the Bat 3 mRNA thus preventing its translation into protein. The Bat 3 mRNA ribozymes can be chemically synthesized to selectively inhibit Bat 3 production. In addition, the ribozymes may be chemically modified allowing the ribozymes to be more stable and active. Included are also ribozymes that not only cleave Bat 3-specific RNA molecules but also form carbon-carbon bonds in a covalent fashion, which raises the possibility of ribozymes that can catalyze other types of chemical reactions.

In a further embodiment of the present invention the translation of the Bat 3 gene can be reduced or eliminated by binding of an RNA-binding protein to one or more operator sequences in the 5′-UTR of the Bat 3 mRNA transcript. The bound RNA-binding protein interferes with translation, likely by preventing ribosome assembly or blocking the movement of the ribosome along the transcript from 5′ to 3′. Such RNA-binding proteins may be multimeric, e.g. dimers of a particular RNA-binding protein. It is also possible within the scope of the present invention that the Bat 3 antagonist inhibits the Bat 3 expression by promoting or at least being involved in the degradation of Bat 3 mRNA.

If the depletion occurs on the protein level, the present invention encompasses antibodies or fragments thereof capable of specifically recognizing one or more epitopes of the Bat 3 gene products, epitopes of conserved variants of the Bat 3 gene products, epitopes of mutant Bat 3 gene products, or peptide fragments of Bat 3 gene products. Such antibodies may include, but are not limited to, polyclonal antibodies, monoclonal antibodies (mAbs), humanized or chimeric antibodies, single-chain antibodies, Fab fragments, F(ab′)2 fragments, Fv fragments, fragments produced by a Fab expression library, anti-idiotypic (anti-Id) antibodies, and epitope-binding fragments of any of the above. The Bat 3 antagonist being an antibody as described above can be used to capture and neutralize Bat 3 in drug-resistant cancer cells. It may be desirable for the present invention if the antibody simultaneously recognizes and neutralizes other proteins than Bat 3. In order to capture and neutralize more proteins, the antibody used as a Bat 3 antagonist can possess more than one specificities, i.e. being, for example, bispecific, trispecific or multispecific.

Epitopes and antigenic regions useful for generating antibodies can be found within the Bat 3 amino acid sequences by procedures available to one of skill in the art. For example, short, unique peptide sequences can be identified in the amino acid sequences that have little or no homology to known amino acid sequences. Preferably the region of a protein selected to act as a peptide epitope or antigen is not entirely hydrophobic; hydrophilic regions are preferred because those regions likely constitute surface epitopes rather than internal regions of the present proteins and polypeptides. These surface epitopes are more readily detected in samples tested for the presence of the present proteins and polypeptides.

Peptides can be made by any procedure known to one of skill in the art, for example, by using in vitro translation or chemical synthesis procedures. Short peptides which provide an antigenic epitope but which by themselves are too small to induce an immune response may be conjugated to a suitable carrier. Suitable carriers and methods of linkage are well known in the art. Suitable carriers are typically large macromolecules such as proteins, polysaccharides and polymeric amino acids. Examples include serum albumins, keyhole limpet hemocyanin, ovalbumin, polylysine and the like. One of skill in the art can use available procedures and coupling reagents to link the desired peptide epitope to such a carrier. For example, coupling reagents can be used to form disulfide linkages or thioether linkages from the carrier to the peptide of interest. If the peptide lacks a disulfide group, one may be provided by the addition of a cysteine residue. Alternatively, coupling may be accomplished by activation of carboxyl groups.

The minimum size of peptides useful for obtaining antigen specific antibodies can vary widely. The minimum size must be sufficient to provide an antigenic epitope which is specific to the protein or polypeptide. The maximum size is not critical unless it is desired to obtain antibodies to one particular epitope. For example, a large polypeptide may comprise multiple epitopes, one epitope being particularly useful and a second epitope being immunodominant.

Antagonists in accordance with the present invention also include polypeptides or peptides which may either directly or indirectly interact with Bat 3 and which elicit inactivation of Bat 3 in an undesired cell population. By polypeptides or peptides which directly interact which Bat 3, those are meant which physically interact with the Bat 3 polypeptide. Whether a polypeptide or peptide is capable of physically interacting with the Bat 3 polypeptide can be tested by techniques well known in the art, such as yeast two-hybrid assays, co-immunoprecipitation assays, Biocore assays and the like. Such assays are also usually well suited for high throughput screening. Accordingly, large libraries of polypeptides or peptides may be screened for suitable antagonists. Moreover, fragments or chemically modified derivatives of said polypeptides or peptides may be used for screening of antagonists. It has been found in accordance with the present invention that the N-terminal portion of the human small glutamine rich TPR-containing protein (hSGT) may serve as a basis for antagonistically acting peptides. Details on the structure of hSGT are to be found in WO2005/016366. Specifically, it has been found that a fragment of hSGT which is C-terminally truncated after the three TRP domains of hSGT is still capable of physically interacting with the Bat 3 polypeptide (e.g., hSGT comprising amino acids 1 to 192). The C-terminal portion of hSGT (e.g., amino acids 81 to 313), however, appears to be dispensable for the interaction with Bat 3. Such an N-terminal hSGT fragment once introduced into a cell shall bind to Bat 3 without eliciting a biologically response. Thus, the fragment shall act antagonistically by acting as a competitive inhibitor. Such an antagonistic fragment, preferably, consists of the TPR and the N-terminal portion of hSGT, more preferably amino acids 1 to 192 of h-SGT as disclosed in WO 2005/016366.

In a further embodiment of the present invention, the Bat 3 antagonist that depletes the expression and/or the function of the Bat 3 can be a so-called aptamer, either a peptide-based aptamer or an oligonucleotide-based aptamer. Peptide aptamers are defined as protein-based recognition agents that consist of constrained combinatorial peptide libraries displayed on the surface of a scaffold protein (e.g. thioredoxin). Peptide aptamers function in trans, interacting with and inactivating gene products without mutating the DNA that encodes them. In principle, combinatorial libraries of peptide aptamers should contain aptamers that interact with any given gene product, thus allowing peptide aptamers to be generated against an organism's entire proteome. Oligonucleotide-based aptamers being used as Bat 3 antagonist according to the present invention comprise DNA as well as RNA aptamers. In this respect, the present invention encompasses also mirror-image L-DNA or L-RNA aptamers, so-called spiegelmers. The aptamers that are also useful as Bat 3 antagonists for the present invention include those which interact with specific proteins and thus prevent or disrupt the specific protein interaction between the Bat 3 and its possible interaction partner.

In the context of the described aptamers, it is also feasible that the Bat 3 antagonist comprises so-called small molecule inhibitors that may exhibit similar properties as aptamers, namely binding to either the Bat 3 or to an interacting partner, thereby inhibiting their proper interaction and, thus, function. The small molecule inhibitor can be a peptide or a small chemical compound, which has been identified by methods known to the skilled artisan, e.g. by computational combinatorial chemistry in combination with screening of compound libraries. Furthermore, the depletion of Bat 3 can be achieved by using so-called muteins of Bat 3. Muteins are derivatives of biologically active proteins the amino acid composition of which has been artificially altered. The muteins can be made via bacterial expression of mutant genes that encode the muteins that have been synthesized from the genes for the parent proteins by oligonucleotide-directed mutagenesis.

Antagonists of Bat 3 also encompass small molecule antagonists. Small molecule antagonists are biologically active small molecules, i.e. molecules which deplete or inactivate Bat 3 in an undesired cell population. A small molecule as referred to in accordance with the present invention may be selected from any class of chemical compounds. Thus, small molecules may be artificial molecules obtained by chemical synthesis or may be naturally occurring molecules. Small molecules referred to in accordance with the present invention, preferably, have a molecular weight of less than 10,000 Da, less than 8,000 Da, less than 6,000 Da, less than 5,000 Da, less than 3,000 Da, less than 2,000 Da or less than 1,000 Da. It is envisaged that the molecule is bioavailable for the cells of the undesired cell population. If the small molecule antagonist shall be applied for in vivo applications, such as the therapeutic applications referred to herein, it is furthermore envisaged that the small molecule shall be biocompatible, too. Artificial small molecule antagonists can be obtained by screening of chemical libraries using methods elsewhere described in this specification. A chemical library as referred to herein shall include searchable populations of small molecules or mixtures of molecules. In one embodiment, the library is comprised of samples or test fractions (either mixtures of small molecules or isolated small molecules) which are capable of being screened for activity. A chemical library in accordance with the present invention may comprise all classes of artificial chemical compounds. Preferably, however, the chemical compounds should at least be bioavailable and, more preferably, biocompatible. Within the last decade, small molecule libraries have been generated using combinatorial chemistry techniques. Naturally occurring small molecules may be naturally occurring small molecules such as primary or secondary metabolites. Such naturally occurring small molecules can be obtained from any cell, tissue, organ or organism by standard purification and fractioning techniques, e.g., chromatography based techniques like HPLC. The naturally occurring small molecules may also be comprised by a chemical library which can be screened as described elsewhere in this specification.

The person skilled in the art is aware of a variety of methods how to introduce the disclosed Bat 3 antagonists into the target cell. In general, the appropriate method depends on whether the Bat 3 antagonist is a nucleic acid or a peptide. Furthermore, if the Bat 3 antagonist is a peptide it can be delivered into the target cell by introducing the nucleic acid encoding it.

There are several well-known methods of introducing nucleic acids into animal cells, any of which may be used in the present invention and which depend on the host. Typical hosts include mammalian species, such as humans, non-human primates, dogs, cats, cattle, horses, sheep, and the like. At the simplest, the nucleic acid can be directly injected into the target cell/target tissue, or by so-called microinjection into the nucleus. Other methods include fusion of the recipient cell with bacterial protoplasts containing the nucleic acid, the use of compositions like calcium chloride, rubidium chloride, lithium chloride, calcium phosphate, DEAE dextran, cationic lipids or liposomes or methods like receptor-mediated endocytosis, biolistic particle bombardment (“gene gun” method), infection with viral vectors, electroporation, and the like.

For the introduction of the Bat 3 antagonist, respectively the nucleic acid encoding it, into the cell and its expression it can be advantageous if the nucleic acid is integrated in an expression vector. The expression vector is preferably a eukaryotic expression vector, or a retroviral vector, a plasmid, bacteriophage, or any other vector typically used in the biotechnology field. If necessary or desired, the nucleic acid encoding the Bat 3 antagonist can be operatively linked to regulatory elements which direct the transcription and the synthesis of a translatable mRNA in pro- or eukaryotic cells. Such regulatory elements are promoters, enhancers or transcription termination signals, but can also comprise introns or similar elements, for example those, which promote or contribute to the stability and the amplification of the vector, the selection for successful delivery and/or the integration into the host's genome, like regions that promote homologous recombination at a desired site in the genome. For therapeutic purposes, the use of retroviral vectors has been proven to be most appropriate to deliver a desired nucleic acid into a target cell. Preferably, the vectors contain tumor- or tissue specific promoters, or promoters which are specifically activated in the presence of a pathogen.

Particularly advantageous for the present invention is the introduction of the Bat 3 antagonist, particularly the nucleic acids encoding the same, by the use of tumor-specific viruses or engineered viruses the specific targeting of cells of cells (oncotropic viruses, like e.g. parvovirus, viruses which are conjugated to albumin, modified adenovirus, New Castle Disease virus, reovirus, measles virus, recombinant Herpes virus). Other suitable delivery systems include those which allow the specific targeting of tumor cells, e.g. albumin-coated liposomes

The introduction of a nucleic acid encoding an Bat 3 antagonist can be facilitated by the use of so-called translocating peptides. The use of such peptides is particularly preferred for therapeutic purposes. The peptides are usually linked to the nucleic acid to be delivered in a non-covalent manner. In general, peptides are contemplated which mimic and act as efficiently as viruses for gene delivery without their limitations of inducing immune responses or being cytotoxic. Examples of peptides forming peptide-DNA complexes for an efficient delivery into a cell comprise DNA-condensing motifs such as polyamines and modifications thereof, active-targeting motifs such as RGD, endosomolytic peptides such as INF, JTS1 or GALA, and nuclear localization sequences (NLS), e.g. derived from the large tumor antigen of Simian 40 virus. An extensive list of translocating peptides and their proposed delivery mechanisms, all of which are contemplated within the scope of the present invention are described in Morris et al., 2000, Curr. Opin. Biotech., Vol. 8, pages 21 to 27.

If the Bat 3 antagonist is a peptide that shall be directly introduced into the target cell it can be fused to a carrier peptide that mediates the cellular uptake of the peptide. Appropriate carriers are known to the person skilled in the art and include TAT, fibroblast growth factor, galparan (transportan), poly-arginine, and Pep-1. Furthermore, Bat 3 antagonist may be fused to a ligand for a cell surface receptor, or a functional portion thereof, and thus internalized by receptor-mediated endocytosis.

The time period sufficient to allow the Bat 3 antagonist to be effective depends on the nature of the candidate compound, i.e. whether it is a small molecule inhibitor, a nucleic acid, a peptide, a protein or other, as specified in detail supra and has to be determined experimentally. If the Bat 3 antagonist is a siRNA it is preferred that the cell population is cultivated at least 20 hours, more preferably at least 40 hours, most preferred at least 70 hours in order allow the Bat 3 antagonist to be effective.

The Bat 3 antagonist, which is employed for the method of the present invention, namely the depletion of an undesired cell population, can be used for the manufacture of a medicament for the treatment of a disease which is caused by the propagation of an undesired cell population. In a preferred embodiment of the present invention, the disease which is caused by the propagation of an undesired cell population is cancer.

Examples of cancers comprise neuroblastoma, intestine carcinoma such as rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroid carcinoma, papillary thyroid carcinoma, follicular thyroid carcinoma, anaplastic thyroid carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeolid leukemia (AML), chronic myeloid leukemia (CML), adult T-cell leukemia lymphoma, hepatocellular carcinoma, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma and plasmocytoma.

More preferably, the cancer to be treated with a medicament employing a Bat 3 antagonist is selected from the group consisting of cervical carcinoma, neuroblastoma, glioblastoma and breast carcinoma.

In a further embodiment of the present invention, the Bat 3 antagonist, which is employed for the method of the present invention, namely the depletion of an undesired cell population, for example autoreactive cells of the immune system, can be used for the manufacture of a medicament for the treatment of autoimmune diseases.

Examples of such autoimmune diseases are collagen diseases such as rheumatoid arthritis, Lupus erythematodes disseminatus, Sharp syndrome, CREST syndrome (calcinosis, Raynaud syndrome, esophageal dysmotility, teleangiectasia), dermatomyositis, vasculitis (Morbus Wegener) and Sjögren syndrome, renal diseases such as Goodpasture syndrome, rapidly-progressing glomerulonephritis and membrane-proliferative glomerulonephritis type II, endocrine diseases such as type-I diabetes, autoimmune polyendocrinopathy-candidiasis-ectodermal dystrophy (APECED), autoimmune parathyreoidism, pernicious anemia, gonad insufficiency, idiopathic Morbus Addison, hyperthyreosis, Hashimoto thyreoiditis and primary myxedemia, skin diseases such as Pemphigus vulgaris, bullous pemphigoid, Herpes gestationis, Epidermolysis bullosa and Erythema multiforme major, liver diseases such as primary biliary cirrhosis, autoimmune cholangitis, autoimmune hepatitis type-1, autoimmune hepatitis type-2, primary sclerosing cholangitis, neuronal diseases such as multiple sclerosis, Myastenia gravis, myasthenic Lambert-Eaton syndrome, acquired neuromyotony, Guillain-Barré syndrome (Müller-Fischer syndrome), Stiff-man syndrome, cerebellar degeneration, ataxia, opsoklonus, sensoric neuropathy and achalasia, blood diseases such as autoimmune hemolytic anemia, idiopathic thrombocytopenic purpura (Morbus Werlhof), infectious diseases with associated autoimmune reactions such as Malaria and Chagas disease.

The medicaments according to the invention can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, tinctures, sprays or transdermal therapeutic systems, or the inhalative administration in the form of nasal sprays or aerosol mixtures, or, for example, microcapsules, implants or rods. The preferred administration form depends, for example, on the disease to be treated and on its severity.

The preparation of the medicaments can be carried out in a manner known per se. To this end, the Bat 3 antagonist, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human or veterinary medicine.

For the production of pills, tablets, sugar-coated tablets and hard gelatin capsules it is possible to use, for example, lactose, starch, for example maize starch, or starch derivatives, talc, stearic acid or its salts, etc. Carriers for soft gelatin capsules and suppositories are, for example, fats, waxes, semisolid and liquid polyols, natural or hardened oils, etc. Suitable carriers for the preparation of solutions, for example of solutions for injection, or of emulsions or syrups are, for example, water, physiological sodium chloride solution, alcohols such as ethanol, glycerol, polyols, sucrose, invert sugar, glucose, mannitol, vegetable oils, etc. It is also possible to lyophilize the Bat 3 antagonist and to use the resulting lyophilisates, for example, for preparing preparations for injection or infusion. Suitable carriers for microcapsules, implants or rods are, for example, copolymers of glycolic acid and lactic acid.

The pharmaceutical preparations can also contain additives, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

The dosage of the Bat 3 antagonist to be administered, depends on the individual case and is, as is customary, to be adapted to the individual circumstances to achieve an optimum effect. Thus, it depends on the nature and the severity of the disorder to be treated, and also on the sex, age, weight and individual responsiveness of the human or animal to be treated, on the efficacy and duration of action of the compounds used, on whether the therapy is acute or chronic or prophylactic, or on whether other active compounds are administered in addition to the Bat 3 antagonist.

In this respect, the present invention also refers to a method of treating a patient having a disease, preferably cancer or an autoimmune disease, which is caused by the propagation of an undesired cell population, the method comprising introducing an antagonist of Bat 3 into said patient. In a preferred embodiment, the Bat 3 antagonist is a Bat 3-specific siRNA. According to the present invention, the Bat 3 antagonist can, thus, be used for treating a patient having a disease as described supra.

A further embodiment of the present invention refers to a method for screening candidate compounds for at least one Bat 3 antagonist with the ability to inhibit the propagation of a cell population, the method comprising the following steps:
(i) contacting a cell population with a candidate compound, thereby enabling the introduction of said candidate compound into the cells of said cell population,
(ii) cultivating said cell population for a time period sufficient to allow the candidate compound to be effective, and, in parallel, cultivating a control cell population which has not been contacted with the candidate compound, and
(iii) monitoring cell growth and/or cell properties in said cell population and in the control cell population,
   wherein a reduced growth and/or altered cell properties as compared to the control cell population is indicative that the candidate compound is a Bat 3 antagonist which inhibits the propagation of a cell population.

In a preferred embodiment, the method comprises the additional steps:
(iv) qualitatively and/or quantitatively detecting the Bat 3 expression in said cell population and in the control cell population, wherein a lower level of Bat 3 expression is indicative of a compound that is a Bat 3 antagonist, and
(v) determining whether a lower level of Bat 3 expression correlates with a reduced growth and/or altered cell properties of the cell population being contacted with the candidate compound.

The time period sufficient to allow the candidate compound to be effective depends on the nature of the candidate compound, i.e. whether it is a so-called small molecule inhibitor, a nucleic acid, a peptide, a protein or other, and has to be determined experimentally. Ideally, the time period may last at least one cell division cycle in order to verify an effect of the candidate compound on cell growth.

The detecting of the Bat 3 expression can be performed by methods known to the skilled artisan. If the Bat 3 expression is detected on the protein level, methods like Western Blotting, ELISA, RIA, metabolic labelling and immunoprecipitation employing suitable Bat 3-specific antibodies can be performed. If the Bat 3 expression is detected on the nucleic acid level, hybridization techniques, like Southern or Northern Blotting, employing suitable Bat 3-specific probes can be performed. For the detection of Bat 3 expression, it is useful if the detecting molecules, e.g. nucleic acid probes or antibodies, are labelled. Suitable labelling methods/agents include radioactive labelling, fluorescent labelling, attachment of an enzyme moiety the activity of which is measured, attachment of tags (e.g. myc, FLAG, H is, biotin).

The present invention also relates to a method for screening candidate compounds for at least one Bat 3-antagonist with the ability to inhibit the propagation of a cell population comprising:
(i) contacting a Bat 3-polypeptide and a h-SGT polypeptide with a candidate compound for at least Bat 3-antagonist; and
(ii) determining whether the candidate compound is capable of blocking the interaction of Bat 3 and h-SGT, whereby a Bat 3-antagonist is identified.

Preferably, contacting of the Bat 3-polypeptide and the h-SGT polypeptide with the candidate compound for at least one Bat 3-antagonist as referred to in step (i) is carried out under conditions which allow complex formation of the Bat 3-polypeptide and the h-SGT polypeptide. Preferred conditions for an in vitro environment are those which allow for immunoprecipitation as described in the accompanying examples. If the Bat 3-polypeptide and the h-SGT polypeptide are comprised by a cell, it is preferred that the cell is in a mitotic stage. More preferably, a Bat 3-antagonist blocks progression of mitosis of the said cell. Accordingly, in a preferred embodiment, a Bat 3-antagonist can be identified in the method of the present invention by its capability to block progression of mitosis, too. Contacting may, preferably, be carried out in an in vitro environment which allows the formation of protein-protein interactions or in a cellular environment, e.g. within a cell which produces both polypeptides either endogenously or recombinantly. It is to be understood that contacting also comprises allowing the candidate compound to interact with either the Bat 3-polypeptide, the h-SGT polypeptide or both for a time period sufficient to avoid complex formation or to physically separate already existing complexes of Bat 3-polypeptide and h-SGT polypeptide into the “free” (i.e. non-complexed) polypeptides. The Bat 3 polypeptide, the hSGT polypeptide and the candidate compound may be contacted with each other simultaneously or the candidate compound may be brought into contact with a pre-existing mixture of Bat 3 and hSGT polypeptides in already complexed or non-complexed form. Moreover, envisaged by the method of the present invention is that the candidate compound will be contacted with the Bat 3 polypeptide first and subsequently with the hSGT polypeptide or vice versa.

Determining whether the candidate compound is capable of blocking the interaction of Bat 3 and h-SGT can be carried out by measures which allow for the detection of any one of the following species: (a) free Bat 3-polypeptide, (b) free h-SGT polypeptide, (c) a Bat 3/h-SGT-complex, (d) a Bat 3/candidate compound complex or (e) a hSGT/candidate complex. Suitable measures comprise immunoprecipitation techniques as described in the accompanying examples using antibodies which specifically recognizing one of the aforementioned species, chromatographic techniques such as size exclusion or affinity chromatography, mass spectrometry such as MALDI TOF mass spectrometry or NMR spectroscopy based techniques. In order to determine whether a candidate compound is capable of blocking the interaction of Bat 3 and hSGT, the normal complex formation rate may be determined in a first step, e.g., by measuring the amount of complexes which are formed after a certain time period, in the absence of the candidate compound. In a second step the complex formation rate is determined in the presence of the said candidate compound. A significantly lower complex formation rate, e.g., measured as a reduced amount of complexes formed after the time period, shall be indicative for a Bat 3 antagonist. It is to be understood that the complex formation rate may be alternatively determined by measuring the alteration of the amount of free hSGT or Bat 3 polypeptide over the said time period or by measuring either the formation of Bat 3/candidate compound complexes or hSGT/candidate complexes.

A Bat 3-antagonist as identified by the aforementioned screening method may block the interaction of Bat 3 and h-SGT by physically interacting with the domains of either Bat 3 or h-SGT which are required for the Bat 3/hSGT complex formation (i.e. the interaction domains). Such a Bat 3-antagonist shall competitively block the binding of the two polypeptides. Alternatively, a Bat 3-antagonist may sterically block the interaction of Bat 3 and h-SGT. To this end, it is not necessary that the candidate compound physically interacts with one of the aforementioned interaction domain. Rather, it is sufficient that the antagonist prevents the physical interaction of either the Bat 3 polypeptide molecules with the hSGT polypeptide molecules or vice versa.

A further screening method for a Bat 3 antagonist contemplate by the present invention comprises (i) contacting a candidate compound for a Bat 3 antagonist with the interaction domain of the Bat 3 interaction partner hSGT (ii) determining whether the candidate compound is capable of specifically binding to the said interaction domain. As described elsewhere in this specification, it has been found in the studies underlying the present invention that Bat 3 interaction with hSGT requires the N-terminal portion of hSGT including the TPR, preferably, amino acids 1 to 192 of hSGT. It is to be understood that a candidate compound which specifically binds to said interaction domain will prevent hSGT/Bat 3 complex formation as well and, thus, will act antagonistically with respect to the physiological function of Bat 3. Whether a candidate compound specifically binds to the interaction domain can be determined by various techniques known in the art and, preferably, as described in the accompanying examples.

A preferred Bat 3-antagonist to be identified by the screening method of the present invention is selected from the group consisting of a small molecule, an antibody against a Bat 3-polypeptide, a Bat 3-specific aptamer and a Bat 3-specific mutein. Details on said antagonists are to be found elsewhere in the specification.

The present invention further refers to a method for the preparation of a pharmaceutical composition wherein a Bat 3 antagonist inhibiting the propagation of an undesired cell population is identified according to the screening method as specified above, synthesized in adequate amounts, and formulated into a pharmaceutical composition.

In this respect, the present invention also encompasses a pharmaceutical composition manufactured by identifying a Bat 3 antagonist according to the screening method as specified above, synthesizing it in adequate amounts, and formulating it into a pharmaceutical composition.

The present invention further refers to the use of a Bat 3 antagonist for inhibiting the propagation of an undesired cell population, which is, preferably, in the mitotic stage. In a preferred embodiment, the cell population is a cell population of human cells.

All references cited in this specification are herewith incorporated by reference with respect to their entire disclosure content and the disclosure content specifically mentioned in this specification.

### EXAMPLES

The following Example shall merely illustrate the invention. It shall not be construed, whatsoever, to limit the scope of the invention.

### Example 1

### Flag Tagged hSGT Interacted with Endogenous Hsc70, Hsp70 and Bat 3

HeLa cells (human cervical carcinoma cells) were grown in DMEM (Dulbecco's modified Eagle's medium) and the HeLa cell line H2A-YFP (T. A. Knoch (2002), Approaching the three-dimensional organization of the human genome. Ruperto-Carola University, Heidelberg and K. F. Toth et al., Trichostatin A-induced histone acetylation causes decondensation of interphase chromatin, J Cell Sci 117 (2004), 4277-87) which stably expresses a YFP-tagged histone 2A protein (H2A-YFP) in RPMI-1640 (Roswell Park Memorial Institute) medium both containing 10% Fetal Calf Serum, and maintained in 5% CO₂ and 37° C. NBE and NBK cells (new born kidney epithelial cells) were cultured as described previously (M. Winnefeld, J. Rommelaere, and C. Cziepluch, The human small glutamine-rich TPR-containing protein is required for progress through cell division. Exp Cell Res 293 (2004), 43-57). For co-immunoprecipitation experiments extracts from cells in interphase or prometaphase were used. Interphase cells were enriched by removing mitotic cells through mitotic shake off, whereas prometaphase cells were harvested through mitotic shake off after synchronization by double thymidine treatment (2 mM) followed by an incubation for 10 h in thymidine-free medium containing nocodazole (0.05 μg/ml).

To initiate functional analysis of hSGT in cycling cells at the molecular level, preparative co-immunoprecipitaion experiments were performed with extracts from asynchronous HeLa cell populations expressing Flag-tagged hSGT, using anti-Flag or control antibodies (IVA7). The expression plasmid for Flag tagged hSGT (pXFlaghSGT) was generated through insertion of a BamHI/SalI restriction fragment of hSGT cDNA into the BglII/SalI site of the previously described pXFlag vector (C. Cziepluch et al., Identification of a novel cellular TPR-containing protein, SGT, that interacts with the non-structural protein NS1 of parvovirus H-1, J Virol 72 (1998), 4149-4156). The Flag-tag was employed to avoid competition between antibodies and cellular interaction partners for binding sites within hSGT. Immunoprecipitation was carried out as follows: Cells were lysed by incubation in NETN-buffer (20 mM Tris-HCl, pH 7.5; 100 mM NaCl; 1 mM EDTA; 0.5% NP40), containing 1 mM ADP and protease inhibitor cocktail (Roche), followed by three freezing-thaw cycles. Cell debris was removed through centrifugation (13000 rpm, Haereus Biofuge) at 4° C. for 15 min. Protein containing supernatants were pre-cleared by incubation with protein-A-sepharose C1-4B (Amersham Bioscience) for 20 min at 4° C. Before mixing with pre-cleared extracts, protein A sepharose C1-4B beads were pre-treated in the following manner. Beads were incubated together with either the monoclonal Flag-tag-specific antibody (M2, Sigma) or the control monoclonal antibody (IVA7) (S. Bleker, F. Sonntag and J. A. Kleinschmidt, Mutational analysis of narrow pores at the fivefold symmetry axes of adeno-associated virus type 2 capsids reveals a dual role in genome packaging and activation of phospholipase A2 activity, J Virol 79 (2005), 2528-40) and a rabbit anti mouse antibody IgG (Dianova) at 4° C. for 2 h in NETN-buffer and subsequently all unbound antibodies were removed. Pre-cleared protein extracts and sepharose beads with pre-bound antibodies were incubated for 2 h at 4° C. in buffer A containing 10 mM Tris-HCl, pH 7.5; 100 mM NaCl; 1 mM EDTA; 1 mM ADP and protease inhibitor cocktail (Roche). The unbound proteins were removed and the beads were extensively washed. Bound protein fractions were eluted from the protein-A-sepharose beads by adding 2× Laemmli buffer (U.K. Laemmli, Cleavage of structural proteins during the assembly of the head of bacteriophage T4, Nature 227 (1970), 680-685) containing β-mercaptoethanol (0.5%), and subsequent heat treatment for 5 min at 95° C. For identification of the hSGT interaction partners through colloidal coomassie staining (Invitrogen) and subsequent MALDI-TOF analysis, protein extracts of 4×10⁷ cells were loaded per lane, whereas for immunoblot analysis with specific antibodies against Hsc70, Hsp70, Flag-tag, Hsp90 or Bat 3, protein extracts of 5×10⁶ cells were used. Immunoblotting was essentially performed as previously reported (M. Winnefeld, J. Rommelaere, and C. Cziepluch, The human small glutamine-rich TPR-containing protein is required for progress through cell division, Exp Cell Res 293 (2004), 43-57). In brief, cells were counted, collected through centrifugation, washed in PBS, and boiled 3 min in Laemmli buffer (U.K. Laemmli, Cleavage of structural proteins during the assembly of the head of bacteriophage T4, Nature 227 (1970), 680-685) containing 0.5% β-mercaptoethanol. Samples corresponding to 5×10⁵ cells were fractionated through 10% SDS-PAGE and transferred to a nitrocellulose membrane (Schleicher und Schuell). The membrane was blocked with 10% skimmed milk in PBS/0.4% Tween 20 and incubated with primary antibodies over night. After washing, membranes were incubated for 1 h with horseradish-peroxidase-linked secondary antibodies. After washing, proteins were visualized by enhanced chemiluminescence as recommended by the supplier (Amersham Biosciences). The following primary antibodies were used for detection: AG1.2 for hSGT (M. Winnefeld, J. Rommelaere, and C. Cziepluch, The human small glutamine-rich TPR-containing protein is required for progress through cell division, Exp Cell Res 293 (2004), 43-57); F1/23 for Poly[ADP-ribose] polymerase (PARP) (D. Lamarre et al., Structural and functional analysis of poly(ADP ribose) polymerase: an immunological study, Biochim Biophys Acta 950 (1988), 147-60), M2 for the Flag-tag (Sigma); SPA-820 for Hsc70/Hsp70 (Stressgen), 16F1 for Hsp90 α and β (Stressgen), Clone DM 1A for α-tubulin (Sigma) and anti-Bat-3 serum for Bat 3. Anti-Bat-3-specific antibodies were raised in rabbits using a (H is)₆-tagged Bat-3 fusion protein as immunogen. The (H is)₆-tagged Bat-3 fusion protein was expressed in Spodoptera frugiperda Sf-9 cells using Invitrogen's BAC-TO-BAC Baculovirus Expression System, and purified over a Ni-NTA (Qiagen) column.

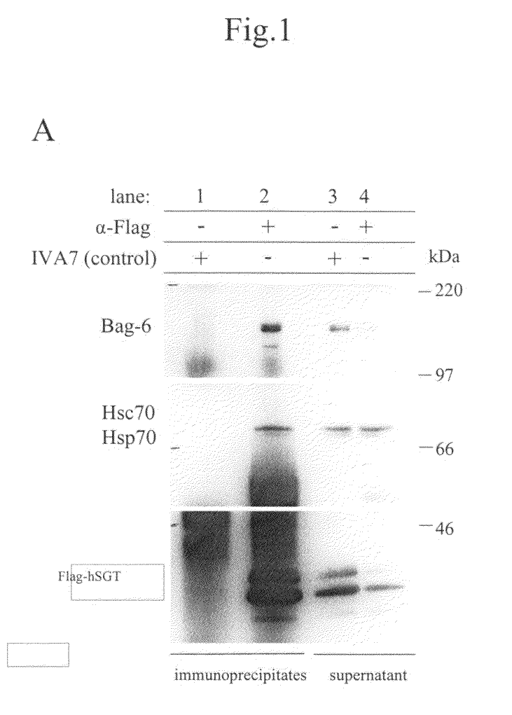

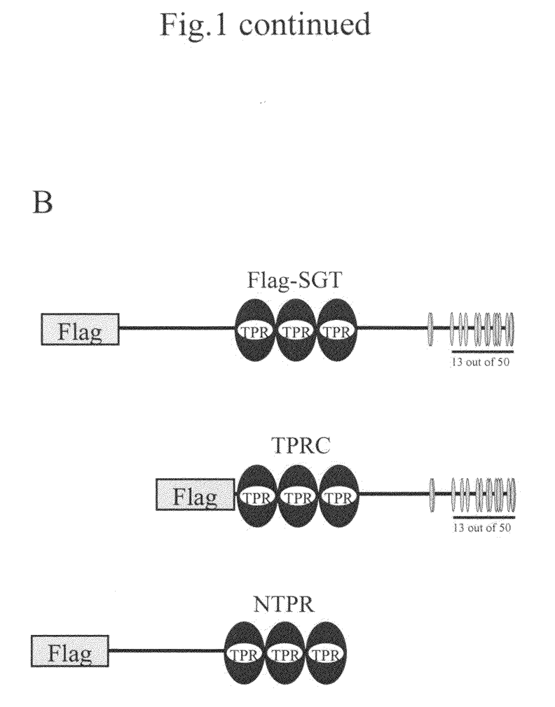

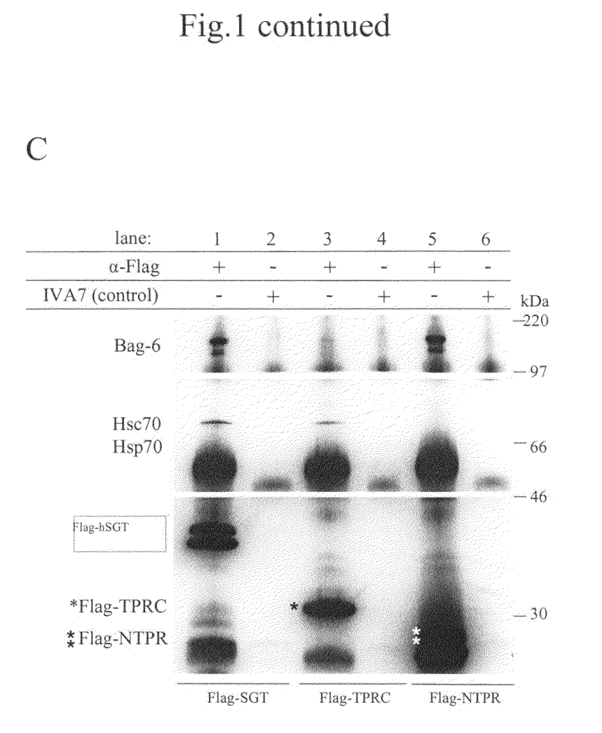

After fractionation of the immune-complexes through SDS-PAGE, all polypeptide species which were detectable after colloidal coomassie-staining and which did not correspond to Flag-tagged hSGT polypeptides or immunglobulines were identified through subsequent MALDI-TOF mass spectrometry. Thereby, Bat 3 and Hsp70, Hsc70 were identified (supplemental data -3.1/3.2-, -4.1/4.2-). These candidates were verified as bona fide interaction partners of hSGT by co-immunoprecipitation and subsequent immuno-blot analysis (FIG. 1A, lane 2). The ratio between precipitated Hsc70 and Hsp70 (FIG. 5A, lane 2) reflected the ratio found in the extracts (FIG. 5A, lane 3 and 4). The capacity of SGT to interact with Hsc70 in vitro has been reported previously (F. H. Liu et al., Specific interaction of the 70-kDa heat shock cognate protein with the tetratricopeptide repeats, J Biol Chem 274 (1999), 34425-34432) and interaction of these proteins was previously demonstrated first in neuronal rat cells where SGT forms a trimeric complex with Hsc70 and the neuron-specific cystein string protein, Csp (S. Tobaben et al., A trimeric protein complex functions as a synaptic chaperone machine, Neuron 31 (2001), 987-999) and later in extracts of rat liver (T. Liou and C. Wang, Small glutamine-rich tetratricopeptide repeat-containing protein is composed of three structural units with distinct functions, Arch Biochem Biophys 435 (2005), 253-63). With respect to Bat 3, results presented here showed the interaction between endogenous Bat 3 and Flag-hSGT for the first time.

In order to assess which fraction of Hsp70 proteins or of Bat 3 was associated with hSGT, equal fractions of the supernatants from the respective co-immunoprecipitation reactions performed with either the Flag (FIG. 1A, lane 4) or the control antibody (FIG. 1A, lane 3) were analyzed by Western blotting. In the case of the Hsp70 proteins, similar amounts appeared to be present in the supernatants of co-immunoprecipitation reaction performed with either Flag-specific or control antibodies, suggesting that only a minor fraction of cellular Hsp70 proteins are associated with hSGT. In contrast however, Bat 3 appeared significantly depleted from the supernatant through the Flag-hsgt-specific co-immunoprecipitation, thereby indicating that a substantial portion of cellular Bat 3 associated with Flag-hSGT.

### Example 2

### The C-Terminus of hSGT is not Essential for Bat 3 Binding while the N-Terminus of hSGT is not Essential for Hsc70 or Hsp70 Binding

It needs to be determined which parts of hSGT were required for interaction with Hsc70, Hsp70 or Bat 3. For these analyses, vectors allowing the expression of Flag-tagged hSGT polypeptides, harbouring truncations either at the N-terminus (TPRC) or at the C-terminus (NTPR) were employed (FIG. 1B). Vectors for the expression of Flag-tagged hSGT deletion mutants NTPR (pXFlagNTPR) and TPRC (pXFlagTPRC) were generated through PCR using the primer pairs: (5′-CGGATCCCCATGGAGACATACAAGTCCAAC-3′ (SEQ ID NO: 7)/5′-GCTCGAGTCAGTTGTCGGGGTCCAGCT-3′(SEQ ID NO: 8)) and (5′-ATGGATCCATGCCCGCGCGAACCG-3′ (SEQ ID NO: 9)/5′-GCTCGAGTCACTCCTGCTGGTCGTCGTTGC-3′ (SEQ ID NO: 10), respectively. Obtained fragments were inserted after BamHI/XhoI restriction into BglII/XhoI sites of pXFlag. Vectors were transfected with Ca-Phosphat (F. L. Graham and A. J. van der Eb, A new technique for the assay of infectivity of human adenovirus 5 DNA, Virology 52 (1973), 456-67).

Previous studies have shown that in vitro interaction of SGT with Hsc70 requires the TPR domain in addition to parts from the C-terminus (F. H. Liu et al., Specific interaction of the 70-kDa heat shock cognate protein with the tetratricopeptide repeats, J Biol Chem 274 (1999) 34425-34432). These findings were in agreement with predictions based on structural data derived from co-crystallization studies of parts of Hsp70 and the Hsp70/Hsp90 organizing protein (Hop) which has a TPR domain very similar to SGT (C. Scheufler, A. Brinker et al., Structure of TPR domain-peptide complexes: critical elements in the assembly of the Hsp70-Hsp90 multichaperone machine, Cell 101 (2000), 199-210). The co-immunoprecipitation experiments with TPRC and subsequent Western blot analysis also revealed that the TPR plus the C-terminal region of hSGT was sufficient for interaction with Hsc70 or Hsp70 (FIG. 1C, lane 3). On the other hand, the presence of the TPR domain alone was not sufficient to allow interaction with Hsc70 or Hsp70, since the N-terminal plus the TPR region of hSGT (NTPR) failed to bind either of these proteins (FIG. 1C, lane 5). The fact that neither Hsc70 nor Hsp70 was co-precipitated with the NTPR mutant showed that the interaction of these proteins with Flag-hSGT was independent of the Flag-tag. In addition, this interaction was not mediated via the previously reported binding of the Hsc70 ATPase domain and the Bag-domain of Bat 3 (K. Thress, J. Song, R. I. Morimoto, and S. Kombluth, Reversible inhibition of Hsp70 chaperone function by Scythe and Reaper, Embo J 20 (2001), 1033-41) which is an NTPR binding partner of hSGT (see below). With respect to Bat 3 interacting domains in hSGT, our results showed that deletion of the C-terminal region of hSGT had no influence on Bat 3 binding (FIG. 1C, lane 5). Therefore, the C terminus was dispensable for hSGT/Bat 3 interaction. However, deletion of the N-terminal region of hSGT strongly reduced interaction with Bat 3 (FIG. 1C, lane 3). It remains to be resolved if the residual weak binding capacity of Bat 3 to the TPRC mutant was mediated either directly through hSGT residues, or indirectly via hSGT bound Hsc70 or Hsp70 proteins. However, the major fraction of Bat 3 co-precipitated with the NTPR mutant in the absence of Hsp70-protein binding and therefore independent of these proteins.

Taken together, these results demonstrated that the identified partners could independently interact with Flag-hSGT and that for Hsc70 or Hsp70 binding on the one hand and Bat 3 binding on the other hand distinct regions within hSGT are dispensable.

### Example 3

### Cell Depleted of Bat 3 Arrested in Mitosis in the Presence of few Mislocalized Chromosomes and Accompanied by Cell Death

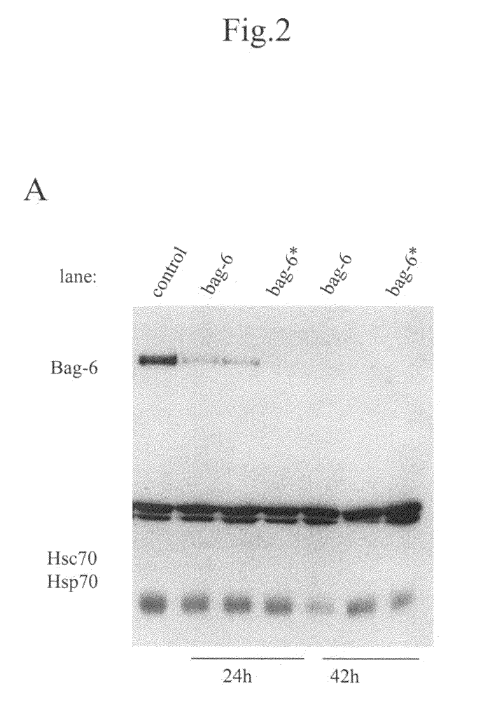

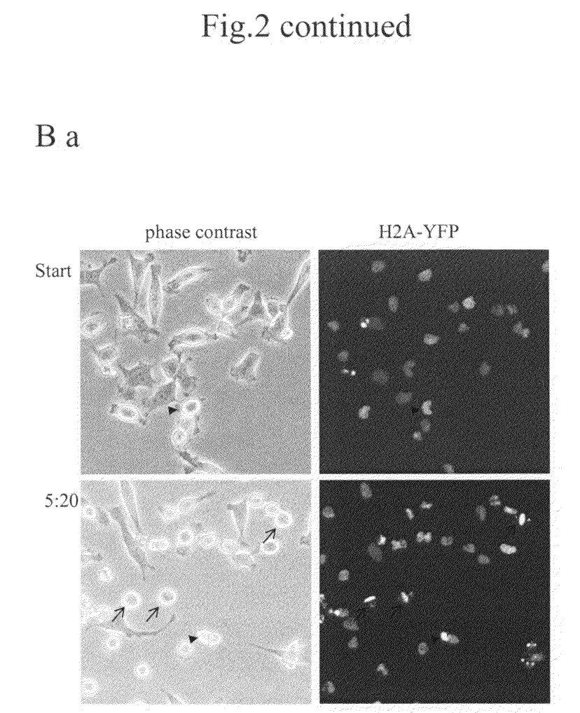

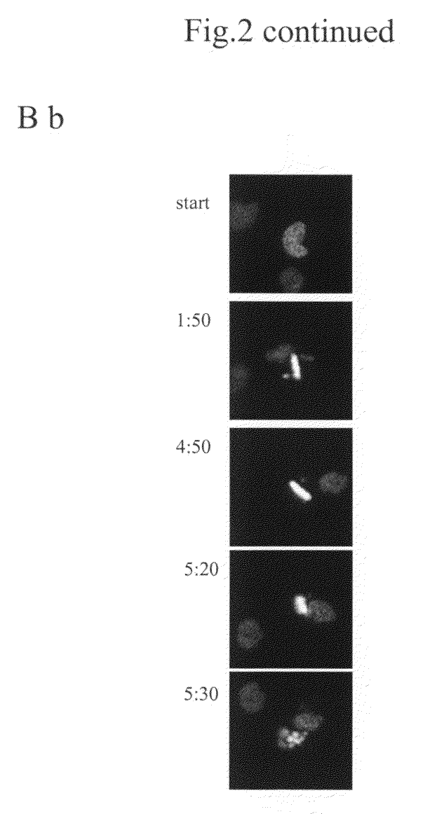

In order to investigate the possible role of Bat 3 at this stage of the cell cycle, the effects of Bat 3 depletion were analyzed. First, it was established that transfection of HeLa cell populations with either of two Bat 3-specific siRNAs (Bat 3 or Bat 3*) led to a significant depletion of Bat 3 protein as detected by Western blotting (FIG. 2A). For Bat 3 depletion, two independent and commercially available oligonucleotides were purchased (Qiagen) (Bat 3 targeted sequence: 5′CAGCTCCGGTCTGATATACAA 3′ (SEQ ID NO: 5) and Bat 3* targeted sequence: 5′ ATGATGCACATGAACATTCAA 3′ (SEQ ID NO: 6)). RNAi-experiments were performed using Oligofectamine reagents (Invitrogen) following the supplier's instructions and cells were analysed at time points as indicated in the figure legends. Silencing efficiency was determined by the residual amount of Bat 3 protein at the indicated time-points post-transfection through immunoblotting.

Time-lapse video microscopy of HeLa H2A-YFP cell populations revealed that Bat 3 depletion led to mitotic arrest of cells which showed persistence of few misaligned chromosomes while all other chromosomes were aligned in the metaphase plate (FIG. 2B a; arrows). After arrest in mitosis, most of these cells died directly in prometaphase without rescuing the misaligned chromosomes (FIG. 2B a cell indicated by filled arrow head and followed over time in FIG. 2B b). These results demonstrated that depletion of either Bat 3 or of hSGT had very similar effects and therefore clearly suggested that hSGT and Bat 3 not only formed complexes during mitosis but also that these proteins contributed together to progress through mitosis. For time-lapse video microscopy, HeLa H2A-YFP cells were seeded in 3 cm dishes at a density of 5×10⁴ cells per dish and treated with Bat 3-specific siRNAs (Bat 3 or Bat 3*) or with gl2 (control) siRNA. At 25 h or 39 h after transfection, dishes were placed on the microscope stage and maintained at 37° C. and 5% CO₂. Cells were time-lapse recorded by fluorescence microscopy (Chroma DiM530; EM 560/40) and by phase contrast microscopy using a Zeiss 10× objective on a Zeiss inverted Axiovert S100TV stand. Fluorescence and phase contrast images (680×680 μm frames) were acquired with a Hamamatsu digital camera at 10 min intervals for up to 16 h using the Openlab software.

1. A method for inhibiting the propagation of an undesired cell population, the method comprising
(i) introducing an antagonist of Bat 3 into at least one cell of said cell population, and
(ii) cultivating said cell population for a time period sufficient to allow said Bat 3 antagonist to be effective, thereby inactivating and/or depleting Bat 3 in said cell population.

2. The method of claim 1, wherein the cell population is in the mitotic stage.

3. The method of claim 1, wherein the cell population is in a resting stage.

4. The method of claim 1, wherein the cell population is a population of human cells.

5. The method of claim 1, wherein the Bat 3 antagonist is selected from the group consisting of a Bat 3-specific siRNA, a transcriptional regulator of the Bat 3 gene, a Bat 3 gene antisense molecule, a Bat 3 mRNA specific ribozyme, an antibody against a Bat 3 polypeptide, a Bat 3-specific aptamer and a Bat 3-specific mutein.

6. The method of claim 5, wherein the Bat 3 antagonist is a Bat 3-specific siRNA.

7. The method of claim 6, wherein the siRNA comprises a sequence as defined by SEQ ID NOs.

8. The method of claim 1, wherein the Bat 3-antagonist is blocking the interaction of Bat 3 and h-SGT.

9. A method of treating a subject suffering from a disease caused by the propagation of an undesired cell population comprising administering to said subject a therapeutically effective amount of a Bat 3 antagonist, optionally in combination with a pharmaceutically active carrier.

10. The method of claim 9, wherein the disease caused by the propagation of an undesired cell population is a cancer disease.

11. The method of claim 10, wherein the cancer disease is selected from the group consisting of neuroblastoma, intestine carcinoma, rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroid carcinoma, papillary thyroid carcinoma, follicular thyroid carcinoma, anaplastic thyroid carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors, glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeolid leukemia (AML), chronic myeloid leukemia (CML), adult T-cell leukemia lymphoma, hepatocellular carcinoma, gall bladder carcinoma, bronchial carcinoma, multiple myeloma, basalioma, teratoma, retinoblastoma, neuroblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma and plasmocytoma.

12. The method of claim 9, wherein the disease caused by the propagation of an undesired cell population is an autoimmune disease.

13. The method of claim 9, wherein the Bat 3 antagonist is selected from the group consisting of a Bat 3-specific siRNA, a transcriptional regulator of the Bat 3 gene, a Bat 3 gene antisense molecule, a Bat 3 mRNA specific ribozyme, an antibody against a Bat 3 polypeptide, a Bat 3-specific aptamer and a Bat 3-specific mutein.

14. The method of claim 9, wherein the Bat 3 antagonist is a Bat 3-specific siRNA.

15. The method of claim 14, wherein the siRNA comprises a sequence as defined by SEQ ID NOs.

16. A method for screening candidate compounds for at least one Bat 3 antagonist with the ability to inhibit the propagation of a cell population, the method comprising the following steps:
(i) contacting a cell population with a candidate compound, thereby enabling the introduction of said candidate compound into the cells of said cell population,
(ii) cultivating said cell population for a time period sufficient to allow the candidate compound to be effective, and parallel cultivating a control cell population which has not been contacted with the candidate compound, and
(iii) monitoring cell growth and/or cell properties in said cell population and in the control cell population,
wherein a reduced growth and/or altered cell properties as compared to the control cell population is indicative that the candidate compound is an Bat 3 antagonist which inhibits the propagation of a cell population.

17. The method of claim 16, the method comprising the additional steps:
(iv) qualitatively and/or quantitatively detecting Bat 3 expression levels in said cell population and in the control cell population, wherein a lower level of Bat 3 expression is indicative of a compound that is a Bat 3 antagonist, and
(v) determining whether a lower level of Bat 3 expression correlates with a reduced growth and/or altered cell properties of the cell population being contacted with the candidate compound.

18. The method of claim 16, wherein the cell population is in the mitotic stage.

19. The method of claim 16, wherein the cell population is a population of human cells.

20. A method for the preparation of a pharmaceutical composition, wherein a Bat 3 antagonist inhibiting the propagation of an undesired cell population is identified according to the method of claim 16, synthesized in adequate amounts, and formulated into a pharmaceutical composition.

21. A method for screening candidate compounds for at least one Bat 3-antagonist with the ability to inhibit the propagation of a cell population comprising:
(i) contacting Bat 3-polypeptide and h-SGT polypeptide with a candidate compound for at least one Bat 3-antagonist; and
(ii) determining whether the candidate compound is capable of blocking the interaction of Bat 3 and h-SGT, whereby a Bat 3-antagonist is identified.

22. The method of claim 21, wherein the Bat 3 polypeptide and h-SGT polypeptide are comprised by a cell.

23. The method of claim 22, wherein the cell is in a mitotic stage.

24. The method of claim 23, wherein a Bat 3 antagonist blocks progression of mitosis.

25. The method of claim 21, wherein the Bat 3-antagonist is selected from the group consisting of: a small molecule, an antibody against a Bat 3 polypeptide, a Bat 3-specific aptamer and a Bat 3-specific mutein.

26. A method for the preparation of a pharmaceutical composition, wherein a Bat 3 antagonist inhibiting the propagation of an undesired cell population is identified according to the method of claim 21, synthesized in adequate amounts, and formulated into a pharmaceutical composition.

27. A method for screening candidate compounds for at least one Bat 3-antagonist with the ability to inhibit the propagation of a cell population comprising:
(i) contacting a candidate compound for a Bat 3 antagonist with the Bat 3 interaction domain of hSGT; and
(ii) determining whether the candidate compound is capable of specifically binding to the said interaction domain.

28. A method for the preparation of a pharmaceutical composition, wherein a Bat 3 antagonist inhibiting the propagation of an undesired cell population is identified according to the method of claim 27, synthesized in adequate amounts, and formulated into a pharmaceutical composition.

29. A medicament comprising a Bat 3 antagonist in a therapeutically effective amount.
